(19) United States
(12) Patent Application Publication
(10) Pub. No.: US 2024/0082544 A1
(43) Pub. Date: Mar. 14, 2024

(54) STEERABLE CATHETER SYSTEM

(71) Applicant: Eindhoven Medical Robotics B.V., Eindhoven (NL)

(72) Inventor: Rolf Iwan Gaasbeek, Eindhoven (NL)

(21) Appl. No.: 18/274,362

(22) PCT Filed: Nov. 17, 2021
(86) PCT No.: PCT/NL2021/050706

§ 371 Date: Jul. 26, 2023

(30) Foreign Application Priority Data
Nov. 17, 2020 (NL) 2026910

(51) Int. Cl. A61M 25/01; A61M 25/00

(52) U.S. Cl. CPC A61M 25/0147; A61M 25/0009; A61M 25/0158; A61M 2025/015

(57) ABSTRACT

The invention deals with a steerable catheter system comprising a polymer shaft with a stiff and bending portion surrounded by a covering tube, where a looped shape memory alloy (SMA) actuator wire is located in lumen along an axis of the shaft at a radial position away from the center of the shaft, where the SMA wire has a looped part at the distal part of the bending portion and two ends on the proximal part of the stiff portion that are connected to connection wires. The two ends at the proximal part are provided with electrically conducting metal clamps that connect the SMA wire to the connection wires, where the clamps are provided with a protrusion that serves as an anchor for the SMA wire and where the covering tube comprises a shrunk heat shrink tube. The clamps with the protrusion serve as an anchor for the SMA wires in the polymer of the shaft. A catheter according to the invention is much more reliable and can also generate larger forces in the SMA wire that lead to larger strokes/bends at the bending portion of the catheter.

Steerable catheter systems are used in Minimal Invasive Systems (MIS) for getting access to difficult to reach places in for instance the human anatomy. For that purpose the distal end of a catheter can bend, so that the catheter can be guided along a complicated route. The catheter comprises a central lumen (hole) so that instruments, like an endoscope or tools, like a cutter or laser can be guided to the tip of the catheter to perform tasks at the difficult to reach place.

The invention deals with a steerable catheter system comprising a polymer shaft with a stiff and bending portion surrounded by a covering tube, where a looped shape memory alloy (SMA) actuator wire is located in lumen along an axis of the shaft at a radial position away from the center of the shaft, where the SMA wire has a looped part at the distal part of the bending portion and two ends that are connected to connection wires.

Such a catheter is known from U.S. Pat. No. 5,897,488 embodiment 16 and FIG. 39, where a looped (bent) SMA wire is used to steer the bending portion of a catheter. An SMA wire reduces in length when heat is applied to the wire. Heat can be applied by leading a current through the SMA wire via the connection wires. In the known catheter three SMA wires are provided in lumen at 120 degrees angles along the axis of the polymer shaft at a radial position away from the center of the shaft. Here along the axis means more or less parallel to this central axis. By applying a current through one of the SMA wires, that SMA wire will decrease in length and the bending portion will bend. Using the three wires gives control in what direction the bend will be, i.e. the distal end of the catheter (tip) can be steered in different directions.

The known catheter has the problem that when using the catheter to get a larger stroke, the bending becomes unpredictable. Thus control of the known catheter is difficult.

Such a catheter is also known from US 2020/289790 A1, FIG. 6.

It is the aim of the invention to provide a catheter that is more reliable when using it at larger strokes and thus have better control possibilities.

According to the invention the two ends are located at the proximal part of the stiff portion and are provided with electrically conducting metal clamps that connect the SMA wire to the connection wires, where the clamps are provided with a protrusion that serves as an anchor for the SMA wire. In the known device the mechanical fixation and the electrical connection are made via caulking. This caulking is rather soft and thus does not provide a good mechanical long term fixation. At larger strokes the forces on the wire become larger and the caulking material creeps, thus making the control of the SMA wire unreliable. The metal clamps are much stronger than the caulking. The clamps also provide a better electrical contact. Moreover the clamps can be easily adapted to accommodate connection wires and SMA wires with different thickness. The protrusion serves as a fixation anchor, so that the location of the clamps does not change even when in operation larger forces are applied via the SMA wire on the bending portion of the catheter. The covering tube comprises a shrunk heat shrink tube. When shrinking the heat shrink tube onto the shaft, the heat shrink tube exerts radial forces on the polymer shaft. This reduces the size of the lumen and fixates the metal clamps by forcing the protrusions into the polymer material of the shaft. The inventive catheter is especially useful when SMA wires with a certain prestress are used. In that case the SMA wire is put under tension and then the heat shrink tube is heated to shrink this tube. A prestressed SMA wire can have much larger decreases in length as compared to a non-prestressed wire. The use of the shrunk tube and the clamps with the protrusion maintain the prestress in the SMA wire after manufacturing. A catheter according to the invention is much more reliable and can also generate larger forces in the SMA wire that lead to larger strokes/bends at the bending portion of the catheter. Preferably the clamps have a cylinder shape with the axis of the cylinder along the axis of the shaft, i.e. parallel to the central axis and the SMA and connection wires are aligned on the axis of the cylinder, where the protrusion is a ring extending around the cylinder. This shape gives a very good electrical contact between SMA wire and the connection wires due to the elongated shape in the axial direction. The ring on the clamp extends around the cylinder in a direction radial to the cylinder's axis, thus providing a very good anchor for the SMA wires.

An even better catheter can be obtained when at the proximal end of the stiff portion a radially extending connection plate is provided with connection holes aligned with the lumen of the shaft and where the material of the connection plate is stronger than the polymer of the shaft at the stiff portion, and where the clamps are located at least partially in the connection holes of the connection plate. The material of the connection plate is stronger than the polymer of the shaft at the stiff portion, i.e. the material has a larger elasticity (Young's) modulus and a higher yield stress than the polymer of the shaft at the stiff portion. The strong connection plate combined with the clamps at least partially in the connection plate provide a very stable basis that can take up large forces without displacement of the clamps in respect to the shaft. The thickness of the plate is not critical as long as the connection plate is thick enough to cope with the forces on the plate. In practice any thickness larger than half the diameter of the shaft is sufficient to take up any forces. The use of a connection plate means the stability and thus the long term reliability of the fixation of the SMA wires is very good.

At smaller bends of the catheter the loop provides enough anchorage for the SMA wire, since the loop also extends in a tangential direction of the shaft. A further advantage is obtained when the SMA wire is provided at the loop with an anchor device of a material that is stronger than the polymer of the shaft at the bending portion, where the anchor device follows and covers an inner curve of the loop. The material of the anchor device is stronger than the polymer of the shaft at the bending portion, i.e. the material has a larger elasticity (Young's) modulus and a higher yield stress than the polymer of the shaft at the bending portion. This is especially advantageous when a larger bend of the distal portion of the catheter is required or when prestressed SMA wires are used. Such an anchor device can be a cylinder, where the SMA wire is looped around in a radial direction. The cylinder then supports the inner loop of the SMA wire. Preferably the anchor device has a U-shape. This shape has the advantage that the legs of the U can be located in the lumen of the shaft, thus fixing the position of the loop precisely. The U-shaped and cylinder anchor devices make it possible to give the loop a very precisely defined shape. This prevents overstretching in the loop and thus the introduction of microcracks in the SMA wire that can lead to failures. Also when the SMA wire decreases in length and exerts forces on the loop of the SMA wire the cylinder and the U-shaped device re-enforce the loop, so that the shape of the SMA wire at the loop does not change. This increases the reliability of the system. Moreover when the SMA wire is heated, the cylinder and the U-shaped anchor device also provide a larger surface so that the local stresses exerted by the loop on the polymer material of the shaft are reduced. This prevents creep of the loop of the SMA wire in the polymer of the shaft. Such creep reduces the efficiency and reliability of the catheter, since after creep larger reductions in length of the SMA wire are necessary to achieve the same bend of the bending portion of the catheter.

Especially when larger forces are used to get a large bend of the tip of the catheter the behaviour of the catheter will be less reproducible over time. Preferably the anchor device is provided with a radially and/or tangentially extending protrusion that serves as an extra anchor for the loop of the SMA wire. The protrusion even further enhances the surface between SMA and polymer of the shaft, thus further reducing stresses and increasing reliability. The protrusion can be for instance in the form of rings around the legs of the u-shaped anchor device, or in the form of a radially and/or tangentially extending protrusion. The directions radially and tangentially are taken in respect of the shaft. Such a protrusion provides extra grip from the u-shaped device on the polymer of the shaft.

An extra advantage can be obtained when the anchor device is made of an electrically conducting material. That way the loop is not or less heated by the current flowing through the SMA wire since the anchor device electrically shortens the loop of the wire. This has the advantage that the distal end of the bending portion, i.e. the tip of the catheter is not or less heated and there is less possibility that heat is transferred from the tip of the catheter to for instance a patient. Moreover the loop will not change size in an unwanted tangential direction when current flows through the SMA wire.

A further advantage is obtained when at the distal end of the bending portion a radially extending anchor plate is provided with insertion holes aligned with the lumen of the shaft and where the material of this anchor plate is stronger than the polymer of the shaft at the bending portion, and where the SMA wire is provided through the insertion holes and the lumen. The material of the anchor plate is stronger than the polymer of the shaft at the bending portion, i.e. the material has a larger elasticity (Young's) modulus and a higher yield stress than the polymer of the shaft at the bending portion. The stiff anchor plate serves as an anchor for the loop in the wire. Thus the stiff plate prevents deformation and creep of the loop. The thickness of the plate is not critical as long as the anchor plate is thick enough to cope with the forces on the plate. In practice any thickness larger than half the diameter of the shaft is sufficient to take up any forces. The strong anchor plate at the bending portion can be used as an alternative for the cylinder or the u-shaped device but also in combination with such anchor devices. Preferably the loop of the SMA wire is located inside the stiff anchor plate. The SMA wire can be in a u-shaped channel inside the stiff plate or in a groove at the distal surface of the stiff plate, while covered with a coating. This prevents the SMA wire from sticking out and getting damaged. Moreover the SMA wire while heated is shielded by the coating so that is heat does not cause any damage, for instance to a patient.

The invention also deals with a method for manufacturing a steerable catheter system, where a shape memory alloy (SMA) actuator wire is bent at a central portion of the wire so as to provide a loop around a u-shaped metal anchor device provided with a protrusion to anchor the loop in a polymer shaft, where the SMA wire with the u-shaped device is introduced in lumen along the axis of the shaft at a radial position away from the center of the shaft, with the loop part and the anchor device at the distal part of a bending portion of the shaft and with the two ends at the proximal part of a stiff part of the shaft, where the two ends of the SMA wire are electrically connected to connecting wires using metal clamps with protrusions to anchor the ends of the SMA wires in the shaft, where the shaft is covered by a heat shrink covering tube, and where the catheter is heated to shrink the heat tube, while the SMA wire is under tension by pulling on the clamps and anchor device and then the catheter is cooled down to permanently fixate the SMA wire in the shaft.

In an embodiment of the method, the steerable catheter system is a steerable catheter system in accordance with the present invention.

In order to make the stiff and bending portion of the polymer shaft a different polymer can be used at the stiff and bending portion of the shaft. Also polymer tubes of different stiffness around a shaft of the same polymer can be used to get a higher stiffness at the stiff portion than at the bending portion of the shaft. While heating the catheter the SMA wire is kept under tension. The shrinking of the heat shrink tube exerts radial, i.e. compressive forces on the shaft. The high temperature used for the shrinking makes the polymer of the shaft soft. The clamps and the anchor device than shrink into the polymer. After cooling down the polymer of the shaft hardens again and the clamps and the anchor device are securely fixed in the shaft, even when the SMA wire is activated. The SMA wire is kept under tension during the heating, that makes that after cooling the SMA wire is under a prestress. Such a prestress makes that the SMA wire can generate a larger decrease in length, i.e. a larger stroke.

The method can be used to manufacture a catheter with the SMA wire permanently prestressed and fixated within the polymer shaft, so that the catheter is reliable and the properties for controlling the wire do not change while using the catheter.

### DESCRIPTION FIGURES

The invention is further explained with the help of the following drawing in which

FIG. 1 shows a cross-section of a catheter according to the invention along a line B-B′ of FIG. 2,

FIG. 2 shows a radial cross-section along the line A-A′ of FIG. 1 of a catheter according to the invention, where the small striped structures indicated as 12, 60 are in a parallel cross-section at the distal end of the catheter,

FIG. 3 a,b show axial cross-sections with details of clamps for the SMA wire and different diameters electrical connection wires,

FIG. 4 shows details of a connection plate used at a proximal part of the catheter,

FIG. 5 shows a cross-section along line C-C′ of FIG. 2 with details of the anchoring of the loop of the SMA wire using a cylinder-shaped anchor device,

FIG. 6a shows a cross-section along line D-D′ of FIG. 6b (this cross-section is equivalent to the cross-section shown in FIG. 5 for the cylinder) and FIG. 6b shows a cross-section along line E-E′ of FIG. 6a with further details of the anchoring of the loop of the SMA wire using an u-shaped anchoring device, where the striped lines indicate details in a plane parallel to the cross-section,

FIG. 7 shows details of an anchor plate used at a distal part of the catheter.

Directions are indicated with the shaft as a reference. Figures are for reference only and are not drawn to scale.

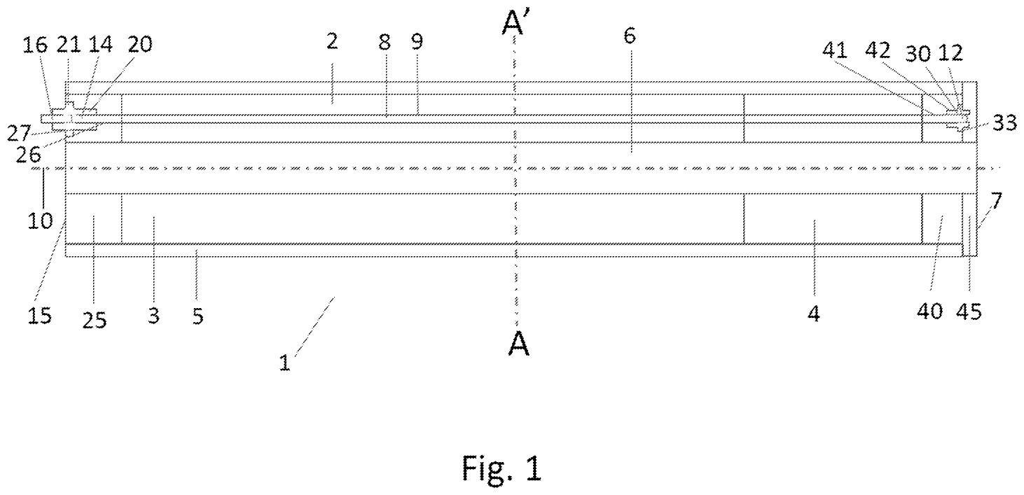

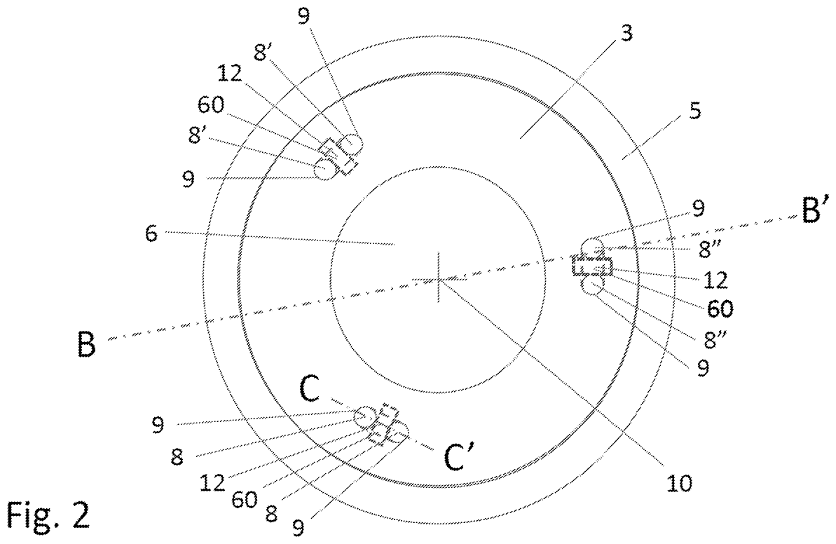

FIGS. 1 and 2 show a steerable catheter system 1 comprising a polymer shaft 2 with a stiff portion 3 and bending portion 4. The polymer shaft 2 is surrounded by a covering tube 5. Inside the polymer shaft 2 is a central hole (lumen) 6 that can be used for guiding an instrument, like an endoscope, laser or scissors to the distal end or tip 7 of the catheter. The lumen 6 can also be used for transferring liquids, like a medicine to the tip 7. A looped SMA actuator wire 8 is located in lumen 9 along an axis 10 of the shaft 2 at a radial position away from the center 10 of the shaft 2. Along the axis means more or less parallel to the central axis 10. The SMA wire 8 has a looped part 12 at the distal part (tip) 7 of the bending portion 4 and two ends 14 on the proximal part 15 of the stiff portion 3. The two ends 14 are connected to connection wires 16.

An SMA wire 8 reduces in length when heat is applied to the wire. Heat can be applied by leading a current through the SMA wire 8 via the connection wires 16.

FIG. 2 shows that three SMA wires 8, 8′ and 8″ are provided in lumen 9 at 120 degrees angles along the axis 10 of the polymer shaft 2 at a radial position away from the central axis 10 of the shaft 2. By applying a current through one of the SMA wires 8, 8′ or 8″, that SMA wire will decrease in length and the bending portion 4 of the catheter 1 will bend. Using the three wires 8, 8′ and 8″ gives control in what direction the bend will be, i.e. the distal end 7 of the catheter 1 can be steered in different directions. The construction of how the wires are located, fixed and electrically connected is the same for all wires 8, 8′ and 8″, so further only the construction of wire 8 will be discussed.

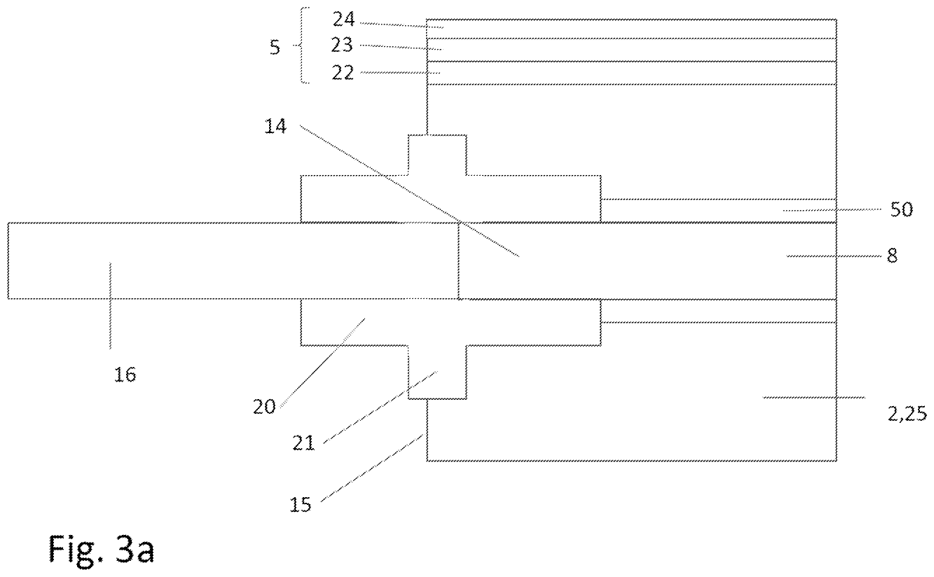

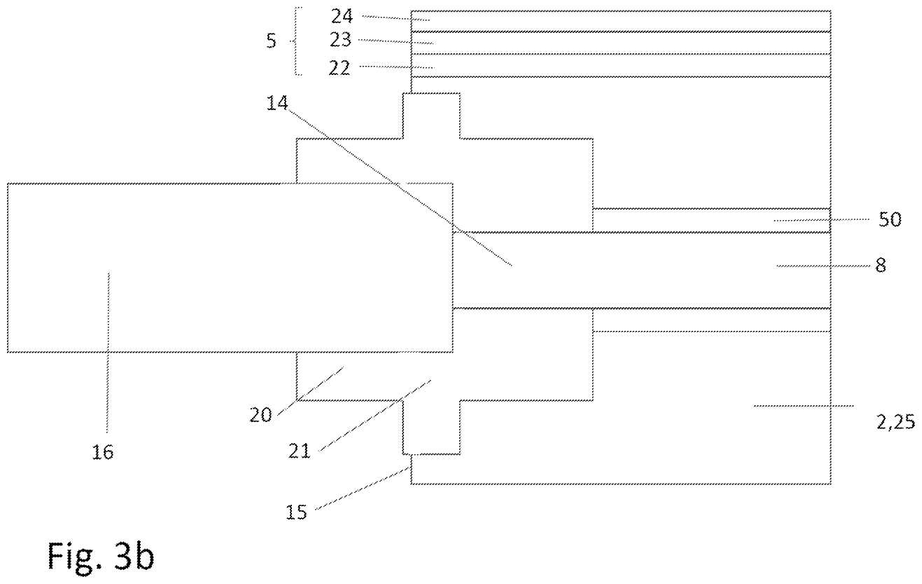

FIG. 3 a, b show how according to the invention the two ends 14 of the SMA wire 8 at the proximal part 15 are provided with electrically conducting metal clamps 20 that connect the SMA wire 8 to connection wires 16, where the clamps are provided with a protrusion 21 that serves as an anchor for the SMA wire 8. FIG. 3a shows an electrical connection wire 16 with the same diameter as the SMA wire 8, whereas FIG. 3b shows an electrical connection wire 16 with double the diameter of the SMA wire 8. The metal clamps 20 can be made of any electrically conducting material, but preferably they are made of nickel or a nickel alloy. Clamps made of this material can be deformed to clamp the SMA wire 8 and the connecting wire 16 strongly together. That way the clamps 20 provide a good electrical connection between wires 8 and 16. The protrusion 21 serves as a fixation anchor, so that the location of the clamps 20 inside the polymer shaft 2 does not change even when in operation larger forces are applied via the SMA wire 8 on the bending portion 4 of the catheter 1. FIG. 3a,b also show the covering tube 5. This covering tube 5 can be made of a shrunk heat shrink tube with different stiffness at the stiff portion 3 and the bending portion 4. FIG. 3a,b show an alternative where an outer covering tube 24 (of same material as shaft 2) is used together with a braiding layer 22 surrounded by a shrunk heat shrink tube 23. The braiding pattern can also be used to further adjust the stiffness of the stiff portion 3 and the bending portion 4. This is done by varying the number of threads used in the braiding layer 22, i.e. making a denser wire mesh at the stiff portion 3 than at the bending portion 4. When shrinking the heat shrink tube 23 in the braiding layer 22, the heat shrink exerts radial forces on the polymer shaft 2. This reduces the size of the lumen 9 and fixates the metal clamps 20 by forcing the protrusions 21 further into the polymer material of the shaft 2. The shaft 2 of catheter 1 can additionally be made of a different polymer in the stiff portion 3 and the bending portion 4, i.e. in the bending portion 4 a polymer with a lower stiffness is used than used for the polymer in the stiff portion 3. The different polymers used in the shaft 2 and the variation in braiding patterns in the braiding layer 22 allow very precise control of the mechanical properties of the catheter 1 in the stiff portion 3 and bending portion 4. The inventive catheter 1 is especially useful when SMA wires 8 with a certain prestress are used. In that case the SMA wire 8 is put under tension while the heat shrink tube 23 in the braiding layer 22 is shrunk by applying heat. A prestressed SMA wire 8 can have much larger decreases in length than a non-prestressed wire 8. The use of the shrunk tube 23 and the clamps 20 with the protrusions 21 maintain the prestress in the SMA wire 8 after manufacturing. The clamps 20 shown in FIG. 3a,b have a cylinder shape with the axis of the cylinder along the axis of the shaft, i.e. parallel to the central axis 10. The SMA wire 8 and connection wires 16 are aligned on the axis of the cylinder, where the protrusion 21 is a radially extending ring around the cylinder. This shape gives a very good electrical contact between SMA wire 8 and the connection wires 16 due to the elongated shape in the axial direction of the shaft 2. The ring 21 on the clamp 20 extends in the radial direction thus providing a very good anchor for the SMA wires 8.

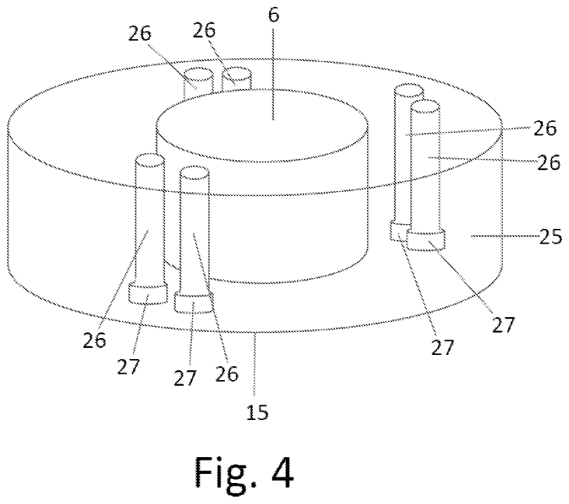

FIGS. 1 and 4 show that an even better catheter 1 can be obtained when at the proximal end 15 of the stiff portion 3 a radially extending connection plate 25 is provided with connection holes 26 aligned with the lumen 9 of the shaft 2. The material of the connection plate 25 is taken stronger than the polymer of the shaft 2 at the location of the stiff portion 3. The clamps 20 are located at least partially in the connection holes 26, of the connection plate 25. The clamps 20 and protrusion 21 are then located in an enlarged part 27 of the connection holes 26 at the proximal part 15, so that the protrusion 21 rests against the material of the connection plate 25. The stiff connection plate 25 combined with the clamps 20 and protrusion 21 at least partially in the connection plate 25 provide a very stable basis that can take up large forces without displacement of the clamps 20 in respect to the shaft 2. The thickness of the plate 25 is not critical as long as the connection plate 25 is thick enough to cope with the forces on the plate 25. The use of a connection plate 25 means the stability and thus the long term reliability of the fixation of the SMA wires 8 is very good. At smaller bends of the bending portion 4, i.e. at larger radii of the distal end of the catheter 1, the loop 12 provides enough anchorage for the SMA wire 8 at the distal end, since the loop 12 also extends in a tangential direction between two lumen 9 used for a wire 8.

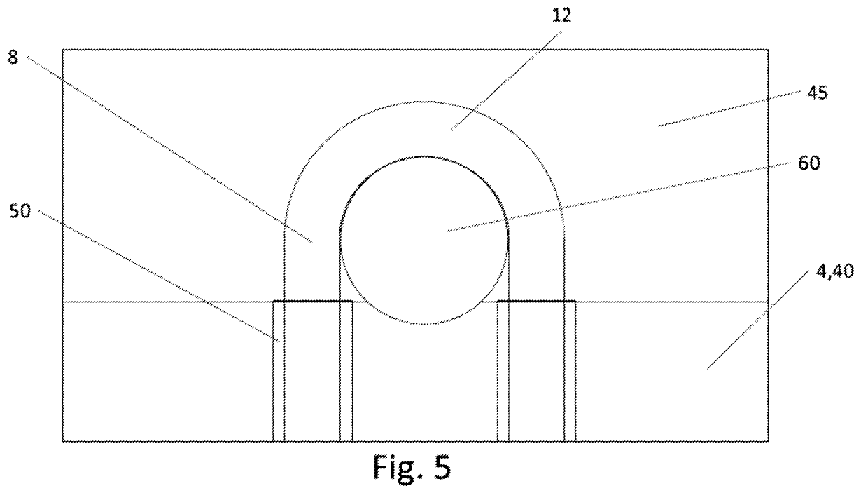

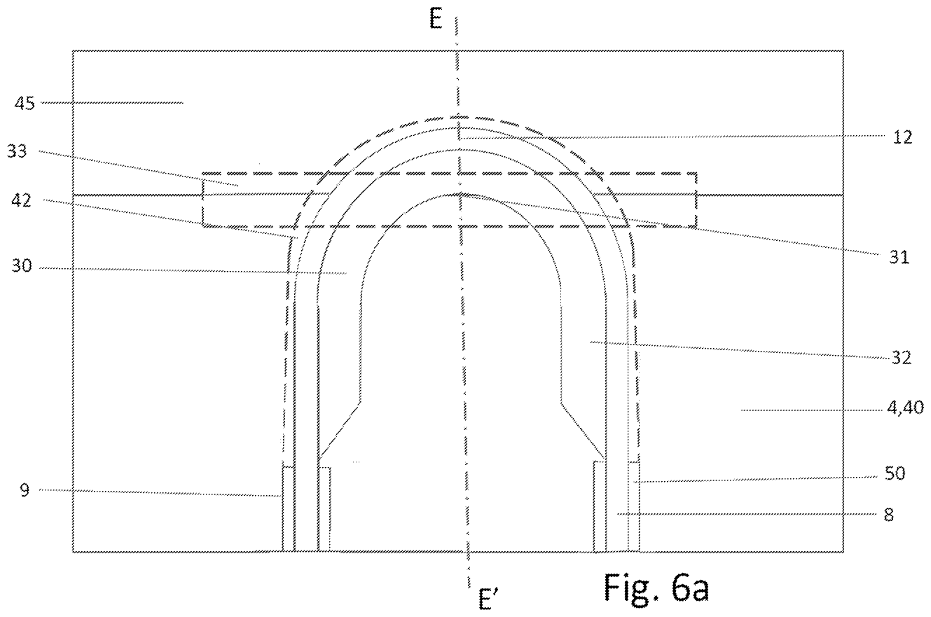

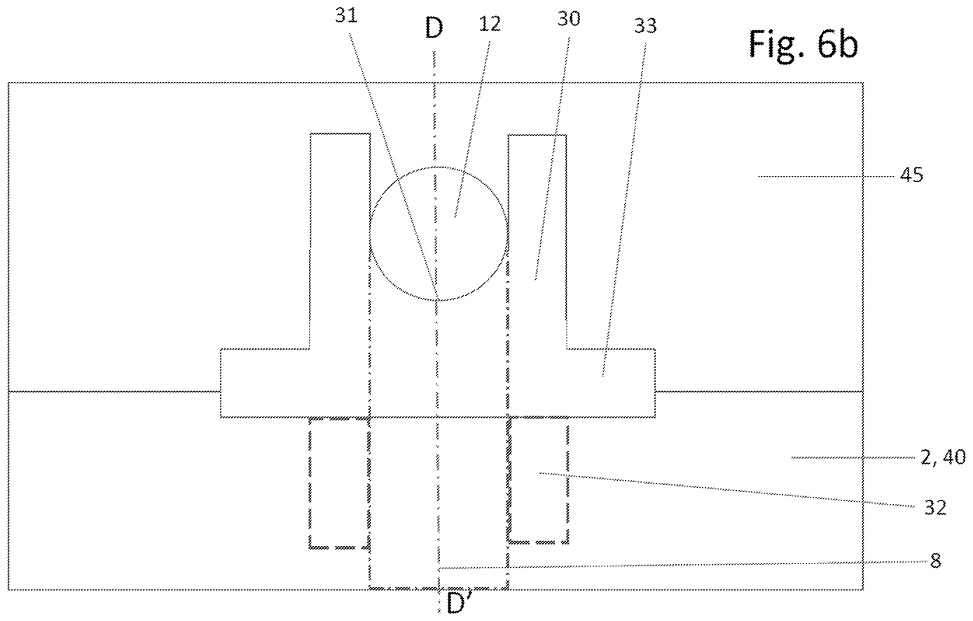

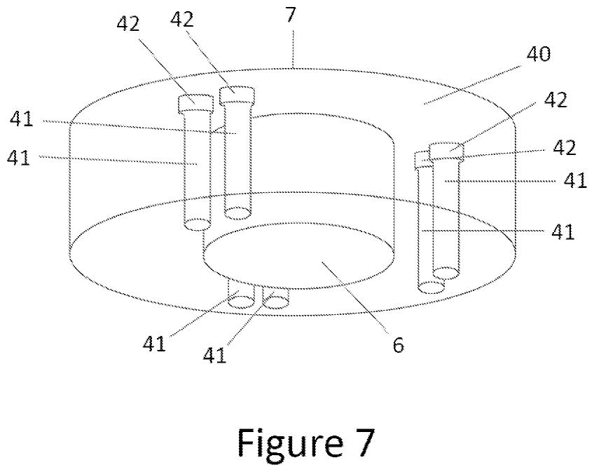

FIGS. 1, 2, 5 and 6 show that preferably the SMA wire 8 is provided at the loop 12 with a stiff anchor device 30, 60 that follows and covers an inner curve 31 of the loop 12. This is especially advantageous when a larger bend of the bending portion 4 of the catheter 4 is required or when prestressed SMA wires 8 are used. FIGS. 2 and 5 shows such one version of an anchor device 60 in the shape of a cylinder with its axis in a radial direction, where the SMA wire 8 is looped around the cylinder 60 in a tangential direction. The cylinder 60 then supports the inner loop 31 of the SMA wire 8, while the extra load-bearing surface provided by the cylinder 60 lowers the stresses in the polymer of the shaft 2. FIG. 6a, b show that preferably the anchor device 30 has a U-shape. This shape has the advantage that the legs 32 of the U can be located in the lumen 9 of the shaft 2, thus fixing the position of the loop 12 precisely. The cylinder 60 and the U-shaped anchor device 30 make it possible to give the loop 12 a very precisely defined shape. This prevents overstretching in the loop 12 and thus prevents the introduction of microcracks in the SMA wire 8 that can lead to failures. Also when the SMA wire 8 decreases in length and exerts forces on the loop 12 of the SMA wire 8 the u-shaped devices 30, 60 re-enforce the loop 12, so that the shape of the SMA wire 8 at the loop 12 does not change. This increases the reliability of the system. Moreover when the SMA wire 8 is heated the anchor devices 30, 60 also provide a larger load-bearing surface so that the local stresses exerted by the loop 12 on the polymer material of the shaft 2 are reduced. This prevents creep of the loop 12 of the SMA wire 8 in the polymer of the shaft 2. Such creep reduces the efficiency and reliability of the catheter 1.

FIG. 6a,b show how the anchor device 30 is provided with a radially and tangentially extending protrusion 33 that serves as an extra anchor for the SMA wire. The protrusion 33 even further enhances the surface between SMA wire 8 and polymer of the shaft 2, thus further reducing stresses and increasing reliability. The protrusion 33 can be for instance in the form of rings around the legs of the u-shaped anchor device 30, or in the form of a radially and tangentially extending protrusion 33 as shown in FIG. 6. Such a protrusion 33 provides extra grip from the u-shaped device 30 on the polymer of the shaft 2.

An extra advantage can be obtained when the anchor devices 30, 60 are made of an electrically conducting material, like a nickel or nickel alloy. That way the loop 12 is not heated by the current flowing through the SMA wire 8 since the anchor device 30,60 electrically shortens the loop 12 of the wire 8. This has the advantage that the distal end 7 of the bending portion 4, i.e. the tip of the catheter 1 is not heated and there is less possibility that heat is transferred from the tip of the catheter 1 to for instance a patient. Moreover the loop 12 will not change size in an unwanted tangential direction when current flows through the SMA wire 8.

FIG. 1, 7 show how an even better anchorage of the loop 12 can be obtained when at the distal end 7 of the bending portion 4 a radially extending anchor plate 40 is provided with insertion holes 41 aligned with the lumen 9 of the shaft 2, 4 and where the material of this anchor plate 40 is stronger than the polymer of the shaft 2, and where the SMA wire 8 is provided through the insertion holes 41 and the lumen 9. The stiff anchor plate 40 serves as an anchor for the loop 12. Thus the strong plate 40 prevents deformation and creep of the loop 12. The thickness of the plate 40 is not critical as long as the anchor plate 40 is thick enough to cope with the forces on the plate 40. The stiff anchor plate 40 at the bending portion 4 can be used as an alternative for the anchor device 30, 60 but it can also be used in combination with an anchor device 30, 60. Preferably the loop 12 of the SMA wire 8 is located inside the stiff anchor plate 40. The SMA wire 8 can be in a u-shaped channel inside the stiff plate 40 or in a groove at the distal surface 44 of the stiff plate 40, while covered with a coating 45. This prevents the SMA wire 8 from sticking out and getting damaged. Moreover the coating 45 provides the SMA wire 8 while heated with an extra shield so that the heat does not cause any damage, for instance to a patient.

The invention also deals with a method for manufacturing a steerable catheter system 1, where a shape memory alloy (SMA) actuator wire 8 is bent at a central portion of the wire 8 so as to provide a loop 12. The loop 12 is formed around a u-shaped device 30. The device 30 comprises a radially and tangentially extending protrusion 33. The SMA wire 8 with the u-shaped device 30 is then introduced in lumen 9 along the axis 10 of a polymer shaft 2 at a radial position away from the center 10 of the shaft 2. The loop part 12 and the u-shaped device 30 are then at the distal part 7 of a bending portion 4 of the shaft 2. The two ends 14 of the SMA wire 8 are at the proximal part 15 of a stiff part 3 of the shaft 2. The two ends 14 of the SMA wire 8 are electrically connected to connecting wires 16 using clamps 20 with protrusions 21 that serve as an anchor for the SMA wires 8 in the shaft 2. The shaft 2 is covered by a braiding layer 22, a permanent heat shrink tube 23 and an outer covering tube 24 made of the same material as the shaft 2. A mandrel is placed in the central lumen 6 and an extra temporary heat shrink tube is applied around the outer covering tube 24. The SMA wire 8 is prestressed by pulling on the clamps 20 and the u-shaped device 30. The whole structure of catheter 1, including the heat shrink tubes is heated, while maintaining the prestress. The extra temporary heat shrink tube provides extra radial compressive forces on the structure. The shrinking of the heat shrink tubes and the high temperature used for the shrinking, force the clamps 20 and the anchor devices 30 to melt in the polymer of the shaft 2, thus providing a very stable anchoring for the SMA wire 8 after cooling down. The outer covering layer 24, the permanent heat shrink tube 23 and the braiding layer 22 fuse together to make a stable structure for catheter 1. The mandrel and the temporary heat shrink tube are removed after the heating process. The method can be used to manufacture a catheter 1 with the SMA wire 8 permanently prestressed and fixated within the polymer shaft 2, so that the catheter 1 is reliable and the properties for controlling the wire 8 do not change while using the catheter 1. The manufacturing method when using a cylinder 60 is identical.

A catheter 1 according to the invention is much more reliable and can also generate larger forces in the SMA wire 8 that lead to larger strokes/bends at the bending portion 4 of the catheter 1.

In a practical example the catheter 1 has a length of 1150 mm, with a stiff portion 3 of 1100 mm and a bending portion 4 of 50 mm. The shaft 2 of the catheter 1 has a diameter of 2.5 mm. The stiff portion 3 is made of a copolymer of polyether and polyamide with a Young's modulus of 510 MPa, a yield stress of 26 MPa and a melting point of 174° C. The bending portion 4 is also made of a copolymer of polyether and polyamide, but a copolymer with a Young's modulus of 18 MPa, a yield stress of less than 4 MPa and a melting point of 144° C. The polymer shaft comprises a central lumen 6 for guiding instruments with a diameter of 1.2 mm. The shaft 2 has six lumen 9 with diameters 0.35 mm. The six lumen are arranged at 120 degree angles as shown if FIG. 2 in pairs of two for one SMA wire 8, the centers of the lumen 9 are located 0.91 mm from the center of the shaft 2. The two lumen 9 for a wire 8 are at a distance of 0.7 mm between the centers of the lumen 9. Such a polymer shaft 2 is made by extrusion. The SMA wires 8 are made of NiTi (Nitinol). The SMA wires have a diameter of 0.15 mm. They are surrounded by thin PTFE tubes 50 (see FIGS. 5 and 6) with a diameter of 0.35 mm and a central lumen of 0.15 mm. The clamps 20 and the protrusion 21 are made of nickel. The clamps 20 have a cylindrical shape with a length of 3 mm and a diameter of 0.35 mm. The protrusions 21 extend radially 0.07 mm from the cylinders of the clamps 20. The protrusions have a thickness of 0.3 mm. Such clamps 20 can be made by using a clamping tool that deforms cylindrical tubes to the desired shape. The connection plate 25 is made of a polyimide polymer with a Young's modulus of 2.5 GPa and a yield stress of 230 MPa. The diameter of the connection plate 25 matches the diameter of the shaft 2 at the proximal part 15. The thickness of plate 25 is 5.5 mm, i.e. approximately 1.5 times the diameter of the shaft 2. In practice any thickness above half the diameter of the shaft 2 proved sufficient to withstand the forces. The diameter of the holes 26 in the connection plate 25 are the same as that of lumen 9. The enlarged part 27 in the connection plate matches the clamps, so that the clamp 20 and the protrusion 21 are half way sunk in the connection plate 25 as shown in FIG. 3. The connection wires 16 are made of copper with a diameter of 0.25 mm. Note that the thickness of the connection wires 16 can be adjusted so as to have a strong wire for connection an external current source to the wire 16. The clamps 20 can be adjusted to accommodate a difference in diameter between SMA wire 8 and connection wire 16. The loop 12 in the SMA wire has an inner radius of 0.55 mm. The cylinder 60 and the u-shaped device 30 match this inner radius. The cylinder 60 of FIG. 2 is made of nickel and has a 0.55 mm diameter and a length of 0.8 mm. The wire 8 is in the middle of the cylinder 60. The cylinder can be sunk halfway in the anchor plate 40. Such a cylinder 60 can be made easily from a round nickel wire with a diameter of 0.55 mm. The preferred u-shaped device 30 is also made of nickel. The device 30 has a length perpendicular to the loop 12 (in a radial direction) of 0.3 mm and the legs 32 extend 0.7 mm. The protrusion 33 extends 0.4 mm in a radial direction. The clamps 20, the cylinder 60 and the u-shaped device 30 can be made in a traditional way, but also using 3d-metal printing. The anchor plate 40 is made of a polyimide polymer with a Young's modulus of 2.5 GPa and a yield stress of 230 MPa. The diameter of the anchor plate 40 matches the diameter of the shaft 2 at the distal part 7. The thickness of plate 40 is 5.5 mm, i.e. approximately 1.5 times the diameter of the shaft 2. In practice any thickness above half the diameter of the shaft 2 proved sufficient to withstand the forces. The diameter of the holes 41 in the anchor plate 40 is the same as that of lumen 9. The enlarged part 42 in the connection plate matches the shape of the u-shaped device 30, so that the u-shaped device 30 and the protrusion 33 are partly sunk in the anchor plate 40. The distal end 7 of the catheter 1 is covered with a coating 45 made of a medical device adhesive, such as Dymax 1072, applied with a thickness of 0.5 mm. In this example a metal braiding is used in the braiding layer 22 to provide the desires stiffness. The braiding 22 is done with a stainless steel wire of 0.03 mm thickness. The number of picks per inch in the braiding layer is varied from 75 in the stiff portion 3 to 60 in the bending portion 3. The braiding layer 22 is surrounded by a permanent heat shrink tube 23 made of polyolefin. Then the permanent heat shrink tube 23 is covered with a tube of the same material as the shaft. Further a temporary heat shrink tube is added. The temporary heat shrink is not critical as long as it provides a rather large shrinkage. A mandrel is placed in the central lumen 6 to prevent shrinkage of this lumen 6. The complete structure is then heated to 220 degrees Celsius, while a relatively high preload (>25N) is applied to the SMA wires 8. After cooling to room temperature the temporary heat shrink tube and the mandrel are removed and the SMA wires 8 have a prestress of approximately 110 MPa. The total diameter of shaft 2 and covering tube 5 after manufacturing is approximately 3.65 mm. The bending stiffness of the stiff portion 3 of the catheter is ˜1000 N/mm² and the bending stiffness at the bending portion 4 is ˜100 N/mm². A catheter according to this embodiment has very good properties. A current of 400 mA through the SMA wire 8 gives a bend of 180 degrees at the distal part 7 of the catheter 1. This does not change with multiple applications of this current through the SMA wire 8, indicating that the catheter is very stable and its control is reliable even after many applications.

1. A steerable catheter system comprising a polymer shaft with a stiff and bending portion surrounded by a covering tube, where a looped shape memory alloy (SMA) actuator wire is located in lumen along an axis of the shaft at a radial position away from the center of the shaft, where the SMA wire has a looped part at the distal part of the bending portion and two ends that are connected to connection wires, where the covering tube comprises a shrunk heat shrink tube, characterized in that the two ends are located at the proximal part of the stiff portion and are provided with electrically conducting metal clamps that connect the SMA wire to the connection wires, where the clamps are provided with a protrusion that serves as an anchor for the SMA wire.

2. The steerable catheter system according to claim 1, where the clamps have a cylinder shape with the axis of the cylinder along the axis of the shaft and the SMA and connection wires are aligned on the axis of the cylinder, where the protrusion is a ring extending around the cylinder.

3. The steerable catheter system according to claim 1, where at the proximal end of the stiff portion a radially extending connection plate is provided with connection holes aligned with the lumen of the shaft and where the material of the connection plate is stronger than the polymer of the shaft at the stiff portion, and where the clamps are located at least partially in the connection holes of the connection plate.

4. The steerable catheter system according to claim 1, where the SMA wire is provided at the loop with an anchor device of a material that is stronger than the polymer of the shaft at the bending portion, where the anchor device follows and covers an inner curve of the loop.

5. The steerable catheter system according to claim 4, where the anchor device has a u-shape.

6. The steerable catheter system according to claim 4, where the anchor device is provided with a radially and/or tangentially extending protrusion that serves as an extra anchor for the loop of the SMA wire.

7. The steerable catheter system according to claim 1, where the anchor device is made of an electrically conducting material.

8. The steerable catheter system according to claim 1, where at the distal end of the bending portion a radially extending anchor plate is provided with insertion holes aligned with the lumen of the shaft and where the material of this anchor plate is stronger than the polymer of the shaft at the bending portion, and where the SMA wire is provided through the insertion holes and the lumen.

9. The steerable catheter system according to claim 8, where the loop of the SMA wire is located inside the strong anchor plate at the bending portion.

10. A method for manufacturing a steerable catheter system, where a shape memory alloy (SMA) actuator wire is bent at a central portion of the wire so as to provide a loop around a u-shaped metal anchor device provided with a protrusion to anchor the loop in a polymer shaft, where the SMA wire with the u-shaped device is introduced in lumen along the axis of the shaft at a radial position away from the center of the shaft, with the loop part and the anchor device at the distal part of a bending portion of the shaft and with the two ends at the proximal part of a stiff part of the shaft, where the two ends of the SMA wire are electrically connected to connecting wires using metal clamps with protrusions to anchor the ends of the SMA wires in the shaft, where the shaft is covered by a heat shrink covering tube, and where the catheter is heated to shrink the heat tube, while the SMA wire is under tension by pulling on the clamps and anchor device and then the catheter is cooled down to permanently fixate the SMA wire in the shaft.

11. The method according to claim 10, wherein the steerable catheter system, wherein the steerable catheter system comprising a polymer shaft with a stiff and bending portion surrounded by a covering tube, where a looped shape memory alloy (SMA) actuator wire is located in lumen along an axis of the shaft at a radial position away from the center of the shaft, where the SMA wire has a looped part at the distal part of the bending portion and two ends that are connected to connection wires, where the covering tube comprises a shrunk heat shrink tube, characterized in that the two ends are located at the proximal part of the stiff portion and are provided with the electrically conducting metal clamps that connect the SMA wire to the connection wires, where the clamps are provided with a protrusion that serves as an anchor for the SMA wire
